(19) United States
(12) Patent Application Publication
(10) Pub. No.: US 2023/0405316 A1
(43) Pub. Date: Dec. 21, 2023

(54) COMPOSITIONS, SYSTEMS, AND METHODS FOR TREATING CANCER USING TUMOR TREATING FIELDS AND VEGF INHIBITORS

(71) Applicant: Novocure Gmbh, Root D4 (CH)

(72) Inventors: Tali Voloshin-Sela, Haifa (IL); Lilach AVIGDOR, Haifa (IL); Anat Klein-Goldberg, Haifa (IL)

(73) Assignee: Novocure Gmbh, Root D4 (CH)

(21) Appl. No.: 18/337,747

(22) Filed: Jun. 20, 2023

Related U.S. Application Data
(60) Provisional application No. 63/353,682, filed on Jun. 20, 2022.
(60) Provisional application No. 63/477,393, filed on Dec. 28, 2022.

(51) Int. Cl. A61N 1/36; A61K 31/47

(52) U.S. Cl. CPC A61N 1/36002; A61K 31/47

(57) ABSTRACT

Compositions, systems, and methods for reducing viability of cancer cells and treating cancer, as well as preventing an increase of volume of a tumor present in a body of a living subject, are disclosed. The systems and methods involve application of an alternating field in combination with administration of at least one composition comprising at least one small molecule anti-angiogenic agent that specifically interacts with VEGF.

### CROSS REFERENCE TO RELATED APPLICATIONS/INCORPORATION BY REFERENCE STATEMENT

The subject application claims benefit under 35 USC § 119(e) of U.S. provisional application No. 63/353,682, filed Jun. 20, 2022, and U.S. provisional application No. 63/477,393, filed Dec. 28, 2022. The entire contents of the above-referenced patent application(s) are hereby expressly incorporated herein by reference.

### STATEMENT REGARDING FEDERALLY SPONSORED RESEARCH OR DEVELOPMENT

Not applicable.

### BACKGROUND

Tumor Treating Fields (TTFields) are low intensity (e.g., 1-3 V/cm) alternating electric fields within the intermediate frequency range (such as, but not limited to, 100-500 kHz) that target solid tumors by disrupting mitosis. This non-invasive treatment targets solid tumors and is described, for example, in U.S. Pat. Nos. 7,016,725; 7,089,054; 7,333,852; 7,565,205; 8,244,345; 8,715,203; 8,764,675; 10,188,851; and 10,441,776. TTFields are typically delivered through two pairs of transducer arrays that generate perpendicular fields within the treated tumor; the electrode arrays that make up each of these pairs are positioned on opposite sides of the body part that is being treated. More specifically, for the OPTUNE® system, one pair of electrodes is located to the left and right (LR) of the tumor, and the other pair of electrodes is located anterior and posterior (AP) to the tumor. TTFields are approved for the treatment of glioblastoma multiforme (GBM), and may be delivered, for example, via the OPTUNE® system (Novocure Limited, St. Helier, Jersey), which includes transducer arrays placed on the patient's shaved head.

Each transducer array used for the delivery of TTFields in the OPTUNE® device comprises a set of ceramic disk electrodes, which are coupled to the patient's skin (such as, but not limited to, the patient's shaved head for treatment of GBM) through a layer of conductive medical gel. The purpose of the medical gel is to deform to match the body's contours and to provide good electrical contact between the arrays and the skin; as such, the gel interface bridges the skin and reduces interference. The device is intended to be continuously worn by the patient for 2-4 days before removal for hygienic care and re-shaving (if necessary), followed by reapplication with a new set of arrays. As such, the medical gel remains in substantially continuous contact with an area of the patient's skin for a period of 2-4 days at a time. In addition, the arrays can be shifted a few centimeters in either direction to allow the skin to heal from one period of treatment to the next. Therefore, a portion of skin that was covered by electrodes/gel for a 2-4 day period could then be uncovered for 2-4 days when the replaced electrodes are shifted slightly; then the device may be reapplied to the original portion of skin for the next 2-4 day period.

Angiogenesis is the process of forming new blood vasculature to increase nutrient and oxygen supply to a region of the body deprived of blood supply. Angiogenesis has been shown to be one of the key processes in the hallmarks of cancer. In addition, vascular endothelial growth factor (VEGF) has been shown to be one of the key players in promoting angiogenesis.

In considering TTFields as a potential improved treatment for osteosarcoma, Oh et al. (Technology in Cancer Research & Treatment (2020) doi:10.1177/1533033820947481) demonstrated that TTFields prevented angiogenesis in human tumor endothelial cells and also downregulated expression of VEGF and matrix metalloproteinase-2 (MMP2). In addition, Tang et al. (J Int Med Res (2012) 40(1):85-94) demonstrated that a reduction in VEGF expression was observed upon exposure to intermediate alternating electric fields in a murine melanoma cell line and a mouse tumor model. Further, Kim et al. (Oncotarget (2016) 7:65125-65136) demonstrated that TTFields inhibit glioblastoma cell migration, invasion, and angiogenesis and asserted that TTFields represent a promising anti-invasion and anti-angiogenesis therapeutic strategy for use in GBM patients.

Despite the availability of Tumor Treating Fields-based therapies, glioblastoma multiforme (GBM) continues to be the most common and aggressive primary malignancy of the central nervous system in adults. The current standard care for recurrent GBM is bevacizumab (AVASTIN®, Genentech, Inc., San Francisco, CA), a humanized monoclonal antibody against vascular endothelial growth factor A (VEGF-A). Emerging preclinical and clinical data indicated that anti-VEGF-A therapies are potentially effective in GBM (Shiyu et al., Biomedicine & Pharmacotherapy (2021) 141:111810). However, patients inevitably develop resistance to bevacizumab and frequently fail to demonstrate significantly better overall survival.

In Ansstas et al. (Case Rep Neurol (2016) 8:1-9), a “pulse dose” approach to bevacizumab administration was combined with TTFields therapy, in which patients with recurrent GBM stopped treatment with bevacizumab, then were treated with TTFields therapy alone, and were subsequently rechallenged with bevacizumab in a “pulse dose” fashion once they became symptomatic and/or had evidence of radiographic progression. The results from this study support the use of TTFields therapy with pulse dose bevacizumab as an option in patients with refractory GBM. In Fallah et al. (J Clin Oncology (2020) 38(15_suppl):2537), the combination of bevacizumab and TTFields was shown to be safe and feasible and to have clinical efficacy in patients with recurrent GBM.

Davidi et al. (Journal of Radiation Oncology (2021) 111 (3):e47-e48), Gkika et al. (Cancers (Basel) (2022) 14(6):1568), and Davidi et al. (Cancers (Basel) (2022) 14(12):2959) examined the addition of TTFields therapy to the treatment of hepatocellular carcinoma (HCC, a highly malignant liver cancer and one of the leading causes of cancer-related mortality worldwide) with sorafenib, a multi-kinase inhibitor and the main first-line treatment for advanced HCC. Combined TTFields/sorafenib treatment exhibited improved response rates when compared to historical controls in patients with advanced HCC.

Jo et al. (Int J Mol Sci (2018) 19(11):3684) studied the effect of sorafenib on the anti-tumor and anti-angiogenesis activities of TTFields in glioblastoma cells and found that the combinatorial treatment inhibited tumor cell motility and invasiveness and angiogenesis.

### BRIEF DESCRIPTION OF THE DRAWINGS

FIG. 1 contains representative images of ovaries with tumors in an orthotropic ovarian cancer mouse model following treatment with TTFields at 200 kHz for 10 days.

FIG. 2 demonstrates that serum circulating VEGF expression is upregulated following treatment with TTFields at 200 kHz for 10 days in the ovarian cancer mouse model.

FIG. 3 demonstrates that serum circulating VEGF expression is upregulated following treatment with TTFields at 150 kHz for 14 days in a lung cancer mouse model.

FIG. 4 demonstrates that intratumoral CD31 expression is upregulated following treatment with TTFields at 200 kHz for 10 days in the ovarian cancer mouse model.

FIG. 5 contains photomicrographs of A2780 cells following TTFields treatment alone, treatment with 4 μM Lenvatinib, and concomitant therapy with both TTFields and 4 μM Lenvatinib.

FIG. 6 demonstrates the effects of concomitant therapy with TTFields and 4 μM Lenvatinib on cell count and apoptosis in the A2780 cell line.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference

All of the compositions, assemblies, systems, kits, and/or methods disclosed herein can be made and executed without undue experimentation in light of the present disclosure. While the compositions, assemblies, systems, kits, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (e.g., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “patient” or “subject” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent/element/method to a patient for therapeutic and/or prophylactic/preventative purposes.

The term “therapeutic composition” or “pharmaceutical composition” as used herein refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof, or an amount of a treatment protocol (e.g., an alternating electric field), sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of at least one condition, disease, and/or infection. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy,” “concomitant therapy,” and “adjunct therapy,” and will be understood to mean that the patient in need of treatment is treated or given another drug for the condition/disease/infection in conjunction with the treatments of the present disclosure. This concurrent therapy can be sequential therapy, where the patient is treated first with one treatment protocol/pharmaceutical composition and then the other treatment protocol/pharmaceutical composition, or the two treatment protocols/pharmaceutical compositions are given simultaneously.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

The term “small molecule” as used herein refers to natural or artificial chemical compounds with well-defined structures that bind to specific biological macromolecules and act as an effector, altering the activity or function of the target to which they bind. Small molecules typically have a molecular weight generally in a range of from about 1 Da to about 1.5 kDa, and their small size allows the molecules to diffuse across cell membranes and reach intracellular sites of action, if necessary. Small molecules are typically smaller than nucleic acids, proteins, enzymes, antibodies, polysaccharides, biologics, and other bio-therapeutic modalities, which are generally more than 1.5 kDa in size.

Turning now to the inventive concept(s), a combinatorial therapy for cancer is disclosed herein. The combinatorial therapy includes the use of alternating electric fields (such as, but not limited to, TTFields) in combination with at least one small molecule anti-angiogenic agent that interacts with either a vascular endothelial growth factor (VEGF; such as, but not limited to, VEGF-A, VEGF-B, VEGF-C, or VEGF-D) or a receptor for VEGF (VEGFR; such as, but not limited to, VEGFR-1, VEGFR-2, or VEGFR-3) and inhibits the interaction between VEGF and VEGFR and thus inhibits a VEGF signaling pathway. The combination of alternating electric fields (e.g., TTFields) with small molecular anti-angiogenic agent(s) provides a synergistic result in the treatment of cancer.

Certain non-limiting embodiments of the present disclosure are directed to a method of reducing viability of cancer cells. The method includes the steps of: (1) administering at least one composition to the cancer cells, wherein the at least one composition comprises at least one small molecule anti-angiogenic agent that specifically and physically interacts with a VEGF or a VEGFR and selectively inhibits the interaction between VEGF and VEGFR and/or inhibits signal transduction by VEGFR; and (2) applying an alternating electric field to the cancer cells for a period of time.

Certain additional non-limiting embodiments of the present disclosure are directed to a method of treating cancer in a subject. The method includes the steps of: (1) administering at least one composition to the subject, wherein the at least one composition comprises at least one small molecule anti-angiogenic agent that specifically and physically interacts with a VEGF or a VEGFR and selectively inhibits the interaction between VEGF and VEGFR and/or inhibits signal transduction by VEGFR; and (2) applying an alternating electric field to a target region of the subject.

Certain additional non-limiting embodiments of the present disclosure are directed to a method of reducing a volume of a tumor present in a body of a living subject, wherein the tumor includes a plurality of cancer cells. The method includes the steps of: (1) administering at least one composition to the subject, wherein the at least one composition comprises at least one small molecule anti-angiogenic agent that specifically and physically interacts with a VEGF or a VEGFR and selectively inhibits the interaction between VEGF and VEGFR and/or inhibits signal transduction by VEGFR; and (2) applying an alternating electric field to a target region of the subject.

Certain additional non-limiting embodiments of the present disclosure are directed to a method of preventing an increase of volume of a tumor present in a body of a living subject, wherein the tumor includes a plurality of cancer cells. The method includes the steps of: (1) administering at least one composition to the subject, wherein the at least one composition comprises at least one small molecule anti-angiogenic agent that specifically and physically interacts with a VEGF or a VEGFR and selectively inhibits the interaction between VEGF and VEGFR and/or inhibits signal transduction by VEGFR; and (2) applying an alternating electric field to a target region of the subject.

The small molecule anti-angiogenic agents utilized in accordance with the present disclosure are defined as specifically and physically interacting with a VEGF or a VEGFR and selectively inhibiting the interaction between VEGF and VEGFR and/or inhibiting signal transduction by VEGFR. By “specifically and physically interacting,” it is meant that there is an actual physical interaction between the small molecule anti-angiogenic agent and a VEGF that inhibits/substantially prevents the VEGF from interacting with a VEGFR, or that there is an actual physical interaction between the small molecule anti-angiogenic agent and a VEGFR that inhibits/substantially prevents the VEGFR from interacting with a VEGF. This “specific interaction” occurs between the specific structures of the small molecule anti-angiogenic agent and the VEGF or VEGFR, as opposed to a “non-specific interaction” that is not dependent on the specific structures of the small molecule anti-angiogenic agent and VEGF/VEGFR. By “selectively inhibiting the interaction,” it is meant that the small molecule anti-angiogenic agent preferentially binds to a VEGF, as determined by an IC₅₀ value, and in a manner that inhibits its ability to interact with its VEGFR, or that the small molecule anti-angiogenic agent preferentially binds to a VEGFR, as determined by an IC₅₀ value, and in a manner that inhibits its ability to interact with its VEGF. However, it is to be understood that the phrases “specifically and physically interacts with a VEGF or a VEGFR” and “selectively inhibits the interaction between VEGF and VEGFR and/or inhibits signal transduction by VEGFR” does not mean that the small molecule anti-angiogenic agent solely binds to/interacts with VEGF or VEGF; indeed, certain small molecule anti-angiogenic agents utilized in accordance with the present disclosure are multi-kinase inhibitors that may also “specifically and physically interact” with other ligands or receptors and “selectively inhibit” the interaction between said ligands/receptors.

In certain particular (but non-limiting) methods, the at least one composition selectively inhibits interaction between at least one VEGF/VEGFR ligand-receptor pair at a half maximal inhibitory concentration (IC₅₀) of less than about 15 nmol/L (i.e., 15 nM), such as, but not limited to, less than about 14 nM, about 13 nM, about 12 nM, about 11 nM, about 10 nM, about 9 nM, about 8 nM, about 7 nM, about 6 nM, about 5 nM, about 4 nM, about 3 nM, about 2 nM, about 1 nM, or the like. In a particular (but non-limiting) embodiment, the at least one composition selectively inhibits interaction between at least one VEGF/VEGFR ligand-receptor pair at a half maximal inhibitory concentration (IC 50) of less than about 10 nmol/L (10 nM).

Steps (1) and (2) of any of the methods of the present disclosure may be performed concomitantly, and in particular, substantially simultaneously or wholly or partially sequentially. When the steps are performed wholly or partially sequentially, the at least one composition comprising at least one small molecule anti-angiogenic agent may be administered before or after application of the alternating electric field has begun.

The methods of the present disclosure may be utilized to treat any types of cancer cells/cancers/tumors that respond to treatment with alternating electric fields (e.g., TTFields) and/or small molecule anti-angiogenic agents. Non-limiting examples of cancer cells/cancers/tumors that can be treated in accordance with the present disclosure include hepatocellular carcinomas, glioblastomas, pleural mesotheliomas, differentiated thyroid cancers, advanced renal cell carcinomas, ovarian cancers, pancreatic cancers, lung cancers, breast cancers, and the like, as well as any combination thereof.

Any type of conductive or non-conductive electrode(s) and/or transducer array(s) that can be utilized for generating an alternating electric field that are known in the art or otherwise contemplated herein may be utilized for generation of the alternating electric field in accordance with the methods of the present disclosure. Non-limiting examples of electrodes and transducer arrays that can be utilized for generating an alternating electric field in accordance with the present disclosure include those that function as part of a TTFields system as described, for example but not by way of limitation, in U.S. Pat. Nos. 7,016,725; 7,089,054; 7,333,852; 7,565,205; 8,244,345; 8,715,203; 8,764,675; 10,188,851; 10,441,776; and 11,452,863; and in US Patent Application Nos. US 2018/0001078; US 2018/0160933; US 2019/0117956; US 2019/0307781; and US 2019/0308016.

The alternating electric field may be generated at any frequency in accordance with the present disclosure. For example (but not by way of limitation), the alternating electric field may have a frequency of about 50 kHz, about 75 kHz, about 100 kHz, about 125 kHz, about 150 kHz, about 175 kHz, about 200 kHz, about 225 kHz, about 250 kHz, about 275 kHz, about 300 kHz, about 325 kHz, about 350 kHz, about 375 kHz, about 400 kHz, about 425 kHz, about 450 kHz, about 475 kHz, about 500 kHz, about 550 kHz, about 600 kHz, about 650 kHz, about 700 kHz, about 750 kHz, about 800 kHz, about 850 kHz, about 900 kHz, about 950 kHz, about 1 MHz, about 2 MHz, about 3 MHz, about 4 MHz, about 5 MHz, about 6 MHz, about 7 MHz, about 8 MHz, about 9 MHz, about 10 MHz, and the like, as well as a range formed from any of the above values (e.g., a range of from about 50 kHz to about 10 MHz, a range of from about 50 kHz to about 1 MHz, a range of from about 50 kHz to about 500 kHz, a range of from about 100 kHz to about 500 kHz, a range of from about 150 kHz to about 300 kHz, etc.), and a range that combines two integers that fall between two of the above-referenced values (e.g., a range of from about 122 kHz to about 313 kHz, a range of from about 78 kHz to about 298 kHz, etc.).

In certain particular (but non-limiting) embodiments, the alternating electric field may be imposed at two or more different frequencies. When two or more frequencies are present, each frequency is selected from any of the above-referenced values, or a range formed from any of the above-referenced values, or a range that combines two integers that fall between two of the above-referenced values.

The alternating electric field may have any field strength in the subject/cancer cells, so long as the alternating electric field is capable of functioning in accordance with the present disclosure. For example (but not by way of limitation), the alternating electric field may have a field strength of at least about 1 V/cm, about 1.5 V/cm, about 2 V/cm, about 2.5 V/cm, about 3 V/cm, about 3.5 V/cm, about 4 V/cm, about 4.5 V/cm, about 5 V/cm, about 5.5 V/cm, about 6 V/cm, about 6.5 V/cm, about 7 V/cm, about 7.5 V/cm, about 8 V/cm, about 9 V/cm, about 9.5 V/cm, about 10 V/cm, about 10.5 V/cm, about 11 V/cm, about 11.5 V/cm, about 12 V/cm, about 12.5 V/cm, about 13 V/cm, about 13.5 V/cm, about 14 V/cm, about 14.5 V/cm, about 15 V/cm, about 15.5 V/cm, about 16 V/cm, about 16.5 V/cm, about 17 V/cm, about 17.5 V/cm, about 18 V/cm, about 18.5 V/cm, about 19 V/cm, about 19.5 V/cm, about 20 V/cm, and the like, as well as a range formed from any of the above values (e.g., a range of from about 1 V/cm to about 20 V/cm, a range of from about 1 V/cm to about 10 V/cm, a range of from about 1 V/cm to about 4 V/cm, etc.), and a range that combines two integers that fall between two of the above-referenced values (e.g., a range of from about 1.1 V/cm to about 18.6 V/cm, a range of from about 1.2 V/cm to about 9.8 V/cm, a range of from about 1.3 V/cm to about 4.7 V/cm, etc.).

The alternating electric field may be applied in a single direction between a pair of arrays or may be alternating in two directions/channels between two pairs of arrays (e.g., front-back and left-right). For example, certain TTFields devices (such as, but not limited to, the OPTUNE® system (Novocure Limited, St. Helier, Jersey)) operate in two directions in order to increase the chances that a dividing cell will be aligned with the electric field such that the electric field can have the desired anti-mitotic effect. However, it will be understood that the scope of the present disclosure also includes the application of the alternating electric field in a single direction. The term “alternating electric field” as used herein will be understood to include application in a single direction/channel as well as in two directions/channels; in addition, the term “alternating electric field” as used herein will be understood to include both application of a single alternating electric field as well as application of a plurality of alternating electric fields in succession for a duration of time.

The alternating electric field may be applied for any continuous or cumulative period of time sufficient to achieve a reduction in viability of cancer cells and/or a reduction in tumor volume (and/or a prevention of increase in tumor volume). The period of time that the alternating electric field is applied includes both a continuous period of time as well as a cumulative period of time. That is, the period of time that the alternating electric field is applied includes a single session (i.e., continuous application) as well as multiple sessions with minor breaks in between sessions (i.e., consecutive application for a cumulative period). For example, a subject is allowed to take breaks during treatment with an alternating electric field device and is only expected to have the device positioned on the body and operational for at least about 60%, at least about 70%, or at least about 80% of the total treatment period (e.g., over a course of one day, one week, two weeks, one month, two months, three months, four months, five months, etc.).

For example, but not by way of limitation, the alternating electric field may be applied for a continuous or cumulative period of time of at least about 1 hour, about 2 hours, about 3 hours, about 4 hours, about 5 hours, about 6 hours, about 7 hours, about 8 hours, about 9 hours, about 10 hours, about 11 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, about 8 days, about 9 days, about 10 days, about 11 days, about 12 days, about 13 days, about 14 days, about 21 days, about 1 month, about 2 months, about 3 months, about 4 months, about 5 months, about 6 months, and the like, as well as a range formed from any of the above values (e.g., a range of from about 1 hour to about 6 months, a range of from about 24 hours to about 72 hours, etc.), and a range that combines two integers that fall between two of the above-referenced values (e.g., a range of from about 14 hours to about 68 hours, etc.).

In a particular (but non-limiting) embodiment, the period of time that the alternating electric field is applied is at least about 24 hours.

Any small molecule anti-angiogenic agents that specifically interact with either a VEGF or a VEGFR and/or that specifically inhibits signal transduction by VEGFR and that are known in the art or are otherwise contemplated herein may be utilized in accordance with the present disclosure, so long as the agents are capable of acting as an anti-angiogenic agent that reduces the formation of new blood vasculature to a cancer cell/tumor, and wherein this anti-angiogenic action occurs through the direct interaction of the molecule with a VEGF or a VEGFR to selectively inhibit the interaction between the VEGF and its respective VEGFR and thereby inhibiting VEGF signaling. Non-limiting examples of small molecular anti-angiogenic agents that may be utilized in accordance with the present disclosure include lenvatinib, axitinib, regorafenib, cabozantinib, anlotinib, pazopanib, albendazole, lucitanib, motesanib, aflibercept, ponatinib, cediranib, tivozanib, telatinib, apatinib, semaxanib, fruquintinib, lucitanib, anlotinib, taxifolin, sulfatinib, dovitinib, ningetinib, AZD2932, LY2874455, MGCD-265 analog, ZM 306416, ZM 323881, KRN 633, YF-452, ODM-203, AEE 788, BMS 605541, MAZ51, Ki 8751, SU 5402, SU 5408, SU5205, SU5214, SU 6668, SU 14813, XL 092, XL 184, BAW2881, BFH772, A-13958, SKLB1002, WAY-340935, ZD-4190, hVEGF-IN-1, R1530, VEGF-Grab, Soluble Vascular Endothelial Growth Factor Decoy Receptor FP3, VEGF decoy receptor fusion protein, a decoy receptor for VEGF, and combinations thereof.

Certain particular (but non-limiting) examples of small molecule agents that can be utilized in accordance with the present disclosure are provided in Table 1, along with their IC₅₀ values for VEGFR-1, VEGFR-2, and/or VEGFR-3.

TABLE 1
VEGFR-1, −2, and −3 IC₅₀ Values for Small Molecule
Anti-Angiogenic Agents

| | IC₅₀ for | | IC₅₀ for | | IC₅₀ for | |
|---|---|---|---|---|---|---|
| Small Molecule | VEGFR-1 | | VEGFR-2 | | VEGFR-3 | |
| Lenvatinib | 22 | nM | 4 | nM | 5.2 | nM |
| Axitinib | 0.1 | nM | 0.2 | nM | 0.1-0.3 | nM |
| Regorafenib | 13 | nM | 4.2 | nM | 46 | nM |
| Cabozantinib | Does not bind | | 0.035 | nM | Does not bind | |
| Anlotinib | Does not bind | | <1 | nM | Does not bind | |
| Pazopanib | 10 | nM | 30 | nM | 47 | nM |
| Albendazole | | | | | | |
| Lucitanib | 7 | nM | 25 | nM | 10 | nM |
| Motesanib | 2 | nM | 3 | nM | 6 | nM |
| Aflibercept | | | | | | |
| Ponatinib | | | 1.5 | nM | | |
| XL092 | | | 1.6 | nM | | |
| R1530 | | | 10 | nM | | |
| sulfatinib | 2 | nM | 24 | nM | 1 | nM |
| SU14813 | 2 | nM | 50 | nM | | |
| Cediranib | 5 | nM | <1 | nM | 3 | nM |
| Dovitinib | 8-13 | nM | 8-13 | nM | 8-13 | nM |
| Tivozanib | 30 | nM | 6.5 | nM | 15 | nM |
| MGCD-265 analog | 3 | nM | 3 | nM | 4 | nM |
| Ki8751 | | | 0.9 | nM | | |
| Ponatinib | | | 1.5 | nM | | |
| Telatinib | | | 6 | nM | 4 | nM |
| ZM 323881 HCl | | | <2 | nM | | |
| Apatinib | | | 1 | nM | | |
| Fruquintinib | 33 | nM | 35 | nM | 0.5 | nM |
| Ningetinib | | | 1.9 | nM | | |
| AZD2932 | | | 8 | nM | | |
| LY2874455 | | | 7 | nM | | |
| BFH772 | | | 3 | nM | | |
| ODM-203 | 26 | nM | 9 | nM | 5 | nM |

In contrast, the previously used sorafenib has IC₅₀ values for VEGFR-1, VEGFR-2, and VEGFR-3 of 21 nM, 90 nM, and 16 nM, respectively. The at least one small molecule anti-angiogenic agents of the present disclosure has an IC₅₀ value for one, two, or three of VEGFR-1, VEGFR-2, and VEGFR-3 of about 10 nM or less, and/or the at least one small molecule anti-angiogenic agents of the present disclosure bind specifically only to (and not simply preferentially to) one of a VEGF or a VEGFR, as described in detail herein. For example, but not by way of limitation, aflibercept is a soluble fusion protein of some of the human extracellular domains of VEGFR-1 and VEGFR-2 and the Fc portion of human immunoglobulin (Ig); as such, aflibercept solely binds to VEGF-A/PIGF.

In certain particular (but non-limiting) embodiments, the at least one small molecule anti-angiogenic agent specifically and physically interacts with one or more of VEGF-A, VEGF-B, VEGF-C, and/or VEGF-D to inhibit a VEGF/VEGFR interaction and VEGFR signaling.

In certain particular (but non-limiting) embodiments, the at least one small molecule anti-angiogenic agent specifically and physically interacts with one or more of VEGFR-1, VEGFR-2, and/or VEGFR-3 to inhibit a VEGF/VEGFR interaction and VEGF signaling. In certain particular (but non-limiting) embodiments, the at least one small molecule anti-angiogenic agent is a Type I, Type II, and/or Type III VEGFR inhibitor. Type I VEGFR inhibitors, also known as ATP competitive inhibitors, generate hydrophobic interactions with the adenine region and form one to three hydrogen bonds with the surrounding residues at the active site of the receptor, thereby competing for binding to the active “DFG-in” conformation in the ATP-binding pocket. Non-limiting examples of Type I VEGFR inhibitors that may be utilized in accordance with the present disclosure include pazopanib, axitinib, ponatinib, motesanib, and the like, as well as any combinations thereof. Type II VEGFR inhibitors are characterized by binding to the inactive “DFG-out” conformation of the kinase and occupying a hydrophobic pocket adjacent to the ATP-binding site. Non-limiting examples of Type II VEGFR inhibitors that may be utilized in accordance with the present disclosure include carbozantinib, lenvatinib, regorafenib, lucitanib, and the like, as well as any combinations thereof. Type III inhibitors, also known as covalent inhibitors, could exert their pharmacological functions through irreversibly binding to cysteines at specific sites on the kinases. Non-limiting examples of Type III VEGFR inhibitors include vatalanib and the like.

The composition comprising small molecule anti-angiogenic agent(s) may be provided with any formulation known in the art or otherwise contemplated herein. In certain particular (but non-limiting) embodiments, the composition comprising at least one small molecule anti-angiogenic agent contains one or more pharmaceutically acceptable carriers (and as such, the composition may also be referred to as a “pharmaceutical composition”). Non-limiting examples of suitable pharmaceutically acceptable carriers include water; saline; dextrose solutions; fructose or mannitol; calcium carbonate; cellulose; ethanol; oils of animal, vegetative, or synthetic origin; carbohydrates, such as glucose, sucrose, or dextrans; antioxidants, such as ascorbic acid or glutathione; chelating agents; low molecular weight proteins; detergents; liposomal carriers; buffered solutions, such as sodium chloride, saline, phosphate-buffered saline, and/or other substances which are physiologically acceptable and/or safe for use; diluents; excipients such as polyethylene glycol (PEG); or any combination thereof. Suitable pharmaceutically acceptable carriers for pharmaceutical formulations are described, for example, in Remington: The Science and Practice of Pharmacy, 23rd ed (2020).

In certain particular (but non-limiting) embodiments, the composition comprising small molecule anti-angiogenic agent(s) may further contain one or more additional active agents. Various active agents utilized in combination with alternating electric fields or small molecule anti-angiogenic agent(s) are known in the art, and certain concomitant therapies are approved by the FDA or currently in clinical trials testing. Non-limiting examples of therapeutic agents that can be utilized in accordance with the present disclosure in combination with small molecule anti-angiogenic agent(s) include anti-PD-1 therapeutics such as (but not limited to) Pembrolizumab, Tislelizumab, Nivolumab, and Cemiplimab; anti-PD-L1 therapeutics such as atezolizumab, avelumab, and durvalumab; chemotherapeutic agents, such as (but not limited to) Paclitaxel, Docetaxel, Ifosamide, Etoposide (Vepesid), Gemcitabine, Lomustine, nab Paclitaxel, temozolomide, and Carboplatin; TKI inhibitors, such as (but not limited to) Everolimus; mTOR inhibitors; Akt inhibitors; PI3K inhibitors; PARP inhibitors; FGF inhibitors; anti-LAB3 agents; anti-CTLA-4 therapeutics; aromatase inhibitors, such as (but not limited to) Letrozole; biologics such as monoclonal antibodies (such as, but not limited to, Denosumab and pembrolizumab); anti-VEGF antibodies or anti-VEGFR antibodies (such as, but not limited to, VEGFR-2 antibodies such as (but not limited to) ramucirumab and DC101); and the like, as well as any combinations thereof.

In certain particular (but non-limiting) embodiments, the small molecule anti-angiogenic agent(s) present in the composition is conjugated to another substance. For example, but not by way of limitation, the small molecule anti-angiogenic agent(s) may be conjugated to a particle or other substance for targeted delivery of the drug to a specific location in the body. In another particular (but non-limiting) embodiment, the composition may comprise small molecule anti-angiogenic agent(s) encapsulated in a nanoparticle.

In addition, any of the compositions of the present disclosure may contain other agents that allow for administration of the compositions via a particular administration route. For example, but not by way of limitation, the compositions may be formulated for administration by oral, topical, transdermal, parenteral, subcutaneous, intranasal, mucosal, intramuscular, intraperitoneal, intravitreal, and/or intravenous routes. Based on the route of administration, the compositions may also contain one or more additional components in addition to the active agent (e.g., small molecule anti-angiogenic agent(s) and/or additional therapeutic agent). Examples of additional secondary compounds that may be present include, but are not limited to, fillers, salts, buffers, preservatives, stabilizers, solubilizers, wetting agents, emulsifying agents, dispersing agents, and other materials well known in the art.

In a particular (but non-limiting) embodiment, the at least one composition comprising the small molecule anti-angiogenic agent(s) is orally administered to the cells/subject/tumor.

The at least one composition comprising small molecule anti-angiogenic agent(s) may be administered before or after application of the alternating electric field has begun. In certain particular (but non-limiting) embodiments, the at least one composition comprising small molecule anti-angiogenic agent(s) may be administered after the application of the alternating electric field has begun. In particular (but not by way of limitation), the at least one composition comprising small molecule anti-angiogenic agent(s) may be administered during application of the alternating electric field (e.g., before the period of time that the alternating electric field is applied has elapsed) and/or after application of the alternating electric field has elapsed.

For example (but not by way of limitation), the at least one composition comprising the small molecule anti-angiogenic agent(s) may be administered after application of the alternating electric field has commenced by a period of at least about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, and the like, as well as a range formed from any of the above values (e.g., a range of from about 24 hours to about 96 hours, etc.), and a range that combines two integers that fall between two of the above-referenced values (e.g., a range of from about 14 hours to about 94 hours, etc.). In a particular (but non-limiting) embodiment, the at least one composition comprising the small molecule anti-angiogenic agent(s) is administered at least about 24 hours after application of the alternating electric field has begun.

In other non-limiting examples, the at least one composition comprising small molecule anti-angiogenic agent(s) may be administered after the period of time that the alternating electric field is applied has elapsed, wherein the at least one composition comprising small molecule anti-angiogenic agent(s) is administered within about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, and the like, of when the period of time elapsed.

In a particular (but non-limiting) embodiment, the at least one composition comprising small molecule anti-angiogenic agent(s) is administered within about 96 hours of when the period of time elapsed.

The composition comprising small molecule anti-angiogenic agent(s) may be administered to the cancer cells/subject at any concentration that provides a therapeutically effective concentration of the small molecule anti-angiogenic agent(s). In certain non-limiting embodiments, the application of the alternating electric field reduces the amount of small molecule anti-angiogenic agent(s) required to be therapeutically effective when compared to a normal therapeutically effective amount administered in the absence of an alternating electric field. For example, but not by way of limitation, the therapeutically effective concentration of small molecule anti-angiogenic agent(s) may be reduced by at least about 25%, about 30%, about 35%, about 40%, about 45%, about 50%, about 55%, about 60%, about 65%, about 70%, about 75% or more with respect to a dosage of small molecule anti-angiogenic agent(s) known to be therapeutically effective in the absence of application of an alternating electric field. In a particular (but non-limiting) embodiment, the therapeutically effective concentration of small molecule anti-angiogenic agent(s) is reduced by at least about 50% when compared to a dosage of small molecule anti-angiogenic agent(s) known to be therapeutically effective in the absence of an alternating electric field.

The therapeutically effective concentration of small molecule anti-angiogenic agent(s) utilized in accordance with the present disclosure may be, for example (but not by way of limitation), about 1 nM, about 2 nM, about 3 nM, about 4 nM, about 5 nM, about 6 nM, about 7 nM, about 8 nM, about 9 nM, about 10 nM, about 11 nM, about 12 nM, about 12.5 nM, about 13 nM, about 14 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 75 nM, about 80 nM, about 85 nM, about 90 nM, about 95 nM, about 100 nM, about 110 nM, about 120 nM, about 130 nM, about 140 nM, about 150 nM, and the like, as well as a range formed from any of the above values (e.g., a range of from about 12.5 nM to about 100 nM, etc.), and a range that combines two integers that fall between two of the above-referenced values (e.g., a range of from about 17 nM to about 83 nM, etc.).

In a particular (but non-limiting) embodiment, the therapeutically effective concentration of small molecule anti-angiogenic agent(s) is from about 12.5 nM to about 100 nM.

In certain particular (but non-limiting) embodiments, the method includes one or more additional steps. For example (but not by way of limitation), the method may further include the step of (3) discontinuing the application of the alternating electric field (such as, but not limited to) to allow the cells/tissue to recover. In addition, any of steps (1) and/or (2) may be repeated one or more times.

In certain particular (but non-limiting) embodiments, the method involves concurrent therapy with two or more compositions. As such, the method may include an additional step of (4) administering at least a second composition to the cancer cells/subject. In a particular (but non-limiting) embodiment, the at least second composition may contain one or more of any of the active substances disclosed or otherwise contemplated herein for use with small molecule anti-angiogenic agent(s).

Various substances and therapies utilized in combination with small molecule anti-angiogenic agent(s) are known in the art, and certain concomitant therapies are approved by the FDA or currently in clinical trials testing. Non-limiting examples of therapeutic agents that can be utilized in accordance with the present disclosure in combination with small molecule anti-angiogenic agent(s) include anti-PD-1 therapeutics such as (but not limited to) Pembrolizumab, Tislelizumab, Nivolumab, and Cemiplimab; anti-PD-L1 therapeutics such as atezolizumab, avelumab, and durvalumab; chemotherapeutic agents, such as (but not limited to) Paclitaxel, Docetaxel, Ifosamide, Etoposide (Vepesid), Gemcitabine, Lomustine, nab Paclitaxel, temozolomide, and Carboplatin; TKI inhibitors, such as (but not limited to) Everolimus; mTOR inhibitors; Akt inhibitors; P13K inhibitors; PARP inhibitors; FGF inhibitors; anti-LAB3 agents; anti-CTLA-4 therapeutics; aromatase inhibitors, such as (but not limited to) Letrozole; biologics such as monoclonal antibodies (such as, but not limited to, Denosumab and pembrolizumab); anti-VEGF or anti-VEGFR antibodies (such as, but not limited to, anti-VEGFR-2 antibodies such as (but not limited to) ramucirumab and DC101); and the like, as well as any combinations thereof.

When present, step (4) may be performed substantially simultaneously or wholly or partially sequentially with the administration of the first composition in step (1), whereby the two separate compositions are administered simultaneously or wholly or partially sequentially. In addition, the two compositions administered in steps (1) and (4) may be administered via the same route (e.g., both orally administered), or the two compositions may be administered by different routes (e.g., one composition orally administered and another composition intravenously administered).

When present, the optional additional administration step (4) may be performed before or after the application of the alternating electric field has begun, during application of the alternating electric field, and/or after application of the alternating electric field has elapsed, in the same manner(s) and time frame(s) as described above for the first composition.

That is, for example (but not by way of limitation), the second composition may be administered after application of the alternating electric field has commenced by a period of at least about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about hours, about 78 hours, about 81 hours, about 84 hours, about 87 hours, about 90 hours, about 93 hours, about 96 hours, about 5 days, about 6 days, about 7 days, and the like, as well as a range formed from any of the above values (e.g., a range of from about 24 hours to about 96 hours, etc.), and a range that combines two integers that fall between two of the above-referenced values (e.g., a range of from about 14 hours to about 94 hours, etc.). In a particular (but non-limiting) embodiment, the second composition is administered at least about 24 hours after application of the alternating electric field has begun.

In other non-limiting examples, the second composition may be administered after the period of time that the alternating electric field is applied has elapsed, wherein the second composition is administered within about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, and the like, of when the period of time elapsed. In a particular (but non-limiting) embodiment, the second composition is administered within about 96 hours of when the period of time elapsed.

In addition, for example (but not by way of limitation), the second composition may be administered after administration of the first substance by a period of at least about 1 minute, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, and the like, as well as a range formed from any of the above values (e.g., a range of from about 24 hours to about 96 hours, etc.), and a range that combines two integers that fall between two of the above-referenced values (e.g., a range of from about 14 hours to about 94 hours, etc.). In a particular (but non-limiting) embodiment, the second composition is administered at least about 12 hours after administration of the first substance.

In certain particular (but non-limiting) embodiments, the method may further comprise the step of (5) administering at least one additional therapy to the cells/subject. Any therapies known in the art or otherwise contemplated herein for use with alternating electric fields (e.g., TTFields) and/or small molecule anti-angiogenic agent therapy may be utilized in accordance with the methods of the present disclosure. Non-limiting examples of additional therapies that may be utilized include radiation therapy, photodynamic therapy, transarterial chemoembolization (TACE), or combinations thereof.

In certain particular (but non-limiting) embodiments, the method may further comprise the step of (6) applying a second alternating electric field to a target region of the subject, wherein the alternating electric field is administered at a different frequency than the frequency of the alternating electric field of step (2). That is, the methods of the present disclosure may comprise applying a first alternating electric field at a first frequency for a first period of time, wherein application of the first alternating electric field at the first frequency for the first period of time increases permeability of cell membranes of the cancer cells; then the at least one composition comprises at least one small molecule anti-angiogenic agent can be administered, and the increased permeability of the cell membranes enables the substance to cross the cell membranes. Then a second alternating electric field can be applied at a second frequency for a second period of time; the second frequency is different from the first frequency, and the second alternating electric field at the second frequency reduces viability of the cancer cells. The use of alternating electric fields at two different frequencies (to affect cell membrane permeability as well as cell viability) is discussed in detail in US Patent Application Publication No. US 2020/0009376, the entire contents of which are hereby expressly incorporated herein by reference.

Any of steps (1) and (2) and optional steps (3), (4), (5), and (6) may be repeated one or more times. Each of the steps can be repeated as many times as necessary. When step (2) is repeated, the transducer arrays may be placed in slightly different positions on the subject than their original placement; relocation of the arrays in this manner may further aid in treatment of the tumor/cancer. In addition, step (1) and optional steps (4) and (5) (when present) of administering compositions/additional therapies may be repeated various times and at various intervals to follow any known and/or generally accepted dosage/treatment regimen for the composition(s)/therapy(ies).

The use of ordinal references to the optional steps is for purpose of example only; the methods of the present disclosure may include one or more of the optional steps (3), (4), (5), and (6) either alone or in combination with one another. That is, the methods of the present disclosure include performing step (3) in the absence of steps (4) or (5), performing step (4) in the absence of steps (3) or (5), performing step (5) in the absence of steps (3) and (4), performing (6) in the absence of (3), (4), and/or (5). In other words, the scope of the methods disclosed herein includes performing steps (1)-(2) (as well as repeating each step as many times as necessary), performing steps (1)-(3) (as well as repeating one or more of steps (1)-(3) as many times as necessary), performing steps (1)-(2) and (4) (as well as repeating one or more of steps (1)-(2) and (4) as many times as necessary), performing steps (1)-(2) and (5) (as well as repeating one or more of steps (1)-(2) and (5) as many times as necessary), performing steps (1)-(4) (as well as repeating one or more of steps (1)-(4) as many times as necessary), performing steps (1)-(3) and (5) (as well as repeating one or more of steps (1)-(3) and (5) as many times as necessary), performing steps (1)-(2) and (4)-(5) (as well as repeating one or more of steps (1)-(2) and (4)-(5) as many times as necessary), performing step (6) in combination with steps (1)-(2), either alone or in combination with one or more of steps (3)-(5), and performing all of steps (1)-(6) (as well as repeating one or more of steps (1)-(6) as many times as necessary).

While the use of concurrent therapy with two substances is explicitly described above, it will be understood that the scope of the present disclosure further includes concurrent therapy with three or more compositions. As such, the method can include one or more additional steps of administering an additional composition to the subject (similar to steps (1) and (4)). Any additional substances administered in the method may be selected from any of the substances disclosed or otherwise contemplated herein for use in combination with small molecule anti-angiogenic agents (as disclosed herein above with respect to optional step (4)); in addition, administration of any additional substances can be performed substantially simultaneously or wholly or partially sequentially with the administration of the first and/or second compositions/substances and in the same manner(s) and time frame(s) as described above for the first and second compositions/substances.

Certain non-limiting embodiments of the present disclosure are related to kits that include any of the components of the alternating electric field (e.g., TTFields) generating systems disclosed or otherwise contemplated herein (such as, but not limited to, one or more transducer arrays and/or one or more hydrogel compositions, as disclosed in U.S. Pat. Nos. 7,016,725; 7,089,054; 7,333,852; 7,565,205; 8,244,345; 8,715,203; 8,764,675; 10,188,851; 10,441,776; and 11,452,863; and in US Patent Application Nos. US 2018/0001078; US 2018/0160933; US 2019/0117956; US 2019/0307781; and US 2019/0308016) in combination with at least one of any of the compositions comprising small molecule anti-angiogenic agent(s) disclosed or otherwise contemplated herein. The kits may optionally further include one or more of any of the optional compositions disclosed or otherwise contemplated herein (such as, but not limited to, one or more optional compositions containing at least one additional active agent). The kits may optionally further include one or more devices (or one or more components of devices) utilized in one or more additional therapy steps.

In a particular (but non-limiting) embodiment, the kit may further include instructions for performing any of the methods disclosed or otherwise contemplated herein. For example (but not by way of limitation), the kit may include instructions for applying one or more components of the alternating electric field (e.g., TTFields) generating device to the skin of the patient, instructions for applying the alternating electric field to the patient, instructions for when and how to administer the composition comprising small molecule anti-angiogenic agent(s) and optionally how to administer one or more optional additional compositions, and/or instructions for when to activate and turn off the alternating electric field in relation to the administration of the composition comprising small molecule anti-angiogenic agent(s) and/or administration of one or more optional compositions and/or therapy steps.

In addition to the components described in detail herein above, the kits may further contain other component(s)/reagent(s) for performing any of the particular methods described or otherwise contemplated herein. For example (but not by way of limitation), the kits may additionally include: (i) components for preparing the skin prior to disposal of the hydrogel compositions and/or transducer arrays thereon (e.g., a razor, a cleansing composition or wipe/towel, etc.); (ii) components for removal of the gel/transducer array(s); (iii) components for cleansing of the skin after removal of the gel/transducer array(s); and/or (iv) and/or (iv) other components utilized with the system (i.e., conductive material, nonconductive material, a soothing gel or cream, a bandage, etc.). The nature of these additional component(s)/reagent(s) will depend upon the particular treatment format, and identification thereof is well within the skill of one of ordinary skill in the art; therefore, no further description thereof is deemed necessary. Also, the components/reagents present in the kits may each be in separate containers/compartments, or various components/reagents can be combined in one or more containers/compartments, depending on the sterility, cross-reactivity, and stability of the components/reagents.

The kit may be disposed in any packaging that allows the components present therein to function in accordance with the present disclosure. In certain non-limiting embodiments, the kit further comprises a sealed packaging in which the components are disposed. In certain particular (but non-limiting) embodiments, the sealed packaging is substantially impermeable to air and/or substantially impermeable to light.

In addition, the kit can further include a set of written instructions explaining how to use one or more components of the kit. A kit of this nature can be used in any of the methods described or otherwise contemplated herein.

In certain non-limiting embodiments, the kit has a shelf life of at least about six months, such as (but not limited to), at least about nine months, or at least about 12 months.

Certain non-limiting embodiments of the present disclosure are related to systems that include any of the components of the alternating electric field generating systems disclosed or otherwise contemplated herein (such as, but not limited to, one or more transducer arrays and/or one or more hydrogel compositions, as disclosed in U.S. Pat. Nos. 7,016,725; 7,089,054; 7,333,852; 7,565,205; 8,244,345; 8,715,203; 8,764,675; 10,188,851; 10,441,776; and 11,452,863; and in US Patent Application Nos. US 2018/0001078; US 2018/0160933; US 2019/0117956; US 2019/0307781; and US 2019/0308016) in combination with at least one of any of the compositions comprising small molecule anti-angiogenic agent(s) disclosed or otherwise contemplated herein. The systems may optionally further include one or more of any of the optional compositions disclosed or otherwise contemplated herein. The systems may optionally further include one or more devices (or one or more components of devices) utilized in one or more additional therapy steps.

### EXAMPLES

Examples are provided herein below. However, the present disclosure is to be understood to not be limited in its application to the specific experimentation, results, and laboratory procedures disclosed herein after. Rather, the Examples are simply provided as one of various embodiments and is meant to be exemplary, not exhaustive.

### Example 1

In contrast to the prior art referenced in the Background section above, which indicated that TTFields have an anti-angiogenic activity, this Example demonstrates that TTFields treatment increases expression of VEGF and actually increases formation of new blood vasculature.

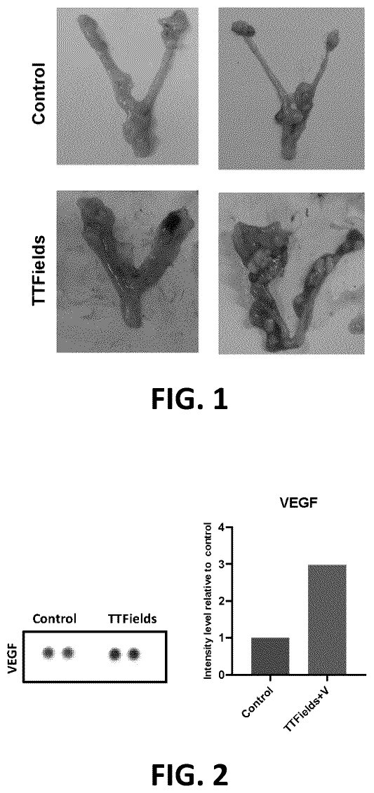

An orthotropic ovarian cancer mouse model was produced by injecting mice with MOSE-L-FFL (Day 0) and confirmed by IVIS at Day 15. Starting on Day 16, the mice were treated with TTFields at 200 kHz (or heat control) for 10 days. As can be seen in FIG. 1, increased vasculature was observed in the tumor-filled ovaries of mice treated with TTFields when compared to untreated control (heat-treated mice).

Mice from this in vivo model of ovarian cancer were sacrificed at Day 26, and blood samples were drawn and collected in designated blood serum tubes centrifuged for 15 minutes in 1000 g and kept at −20° C. Serums were incubated onto cytokine array membranes, and as shown in FIG. 2, an increase in secreted VEGF following TTFields treatment compared to heat-treated mice was observed.

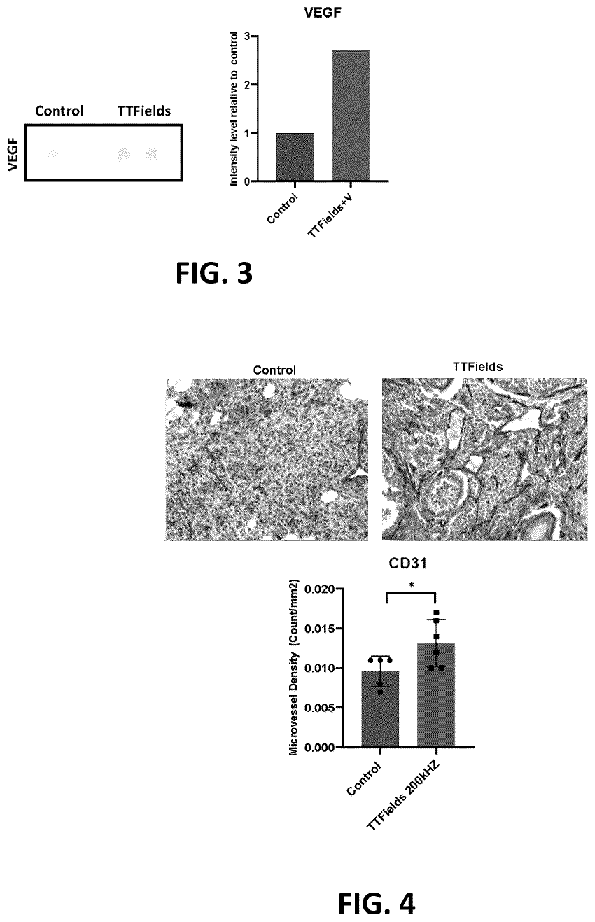

Next, mice with orthotropic lung cancer were produced by injecting mice with LLC-2 (Day and confirmed on Day 7. Starting on Day 7, the mice were treated with TTFields at 150 kHz for 14 days. On Day 21, the mice were sacrificed, and blood samples were drawn and collected in designated blood serum tubes centrifuged for 15 minutes in 1000 g and kept at −20° C. Serums were incubated onto cytokine array membranes, and as shown in FIG. 3, an increase in secreted VEGF following TTFields compared to heat treated mice was observed.

Next, mice from the same in vivo model of ovarian cancer as FIGS. 1-2 were sacrificed on Day 26, and tumor samples were taken for histological analysis of tumor tissue. Tumor tissues were stained by immunohistochemistry for CD31 (a marker for endothelial cells and blood vasculature), and the images were quantified. CD31 staining is indicated by a brown color (DAB staining). As can be seen in FIG. 4, the micro vessel density of TTFields-treated tumors was significantly higher than in the heat-treated control tumors.

Therefore, contrary to the current literature that indicates that TTFields treatment has an anti-angiogenic effect, this Example demonstrates that administration of TTFields actually has a pro-angiogenic effect that results in increased VEGF expression and formation of new blood vasculature to supply the tumors. Thus, administration of at least one small molecule anti-angiogenic agent counteracts the pro-angiogenic effects of TTFields administration in a combinatorial therapy.

### Example 2

Lenvatinib (prescribed as LENVIMA®, Eisai Inc. Nutley, NJ) is a multi-kinase inhibitor of VEGFR1-3. It is clinically used to treat differentiated thyroid cancer (DTC), advanced renal cell carcinoma (RCC), and hepatocellular carcinoma.

This Example examines the combined effect of TTFields and lenvatinib in an effort to understand the impact of the tumor microenvironment and the changes the extracellular matrix (ECM) undergoes following application of TTFields.

The tumor microenvironment, and specifically the ECM components, has been shown to be a crucial part of tumor progression and clinical outcome (Henke et al. (2020) Front Mol Biosci. 6:160; Baghban et al. (2020) Cell Commun Signal, 18:59; Popova et al. (2022) Cancers, 14(1):238). Lenvatinib is a multi-kinase inhibitor that inhibits many of the pathways we have observed to be upregulated upon application of TTFields: three main vascular endothelial growth factor receptors 1, 2, and 3 (VEGFR-1, -2, and -3); fibroblast growth factor receptors 1, 2, 3, and 4 (FGFR-1, -2, -3, and -4); platelet-derived growth factor receptor alpha (PDGFRα); c-Kit; and the RET proto-oncogene. Furthermore, these pathways are known to regulate ECM formation (Nelson et al. (1997) J Vasc Surg, 26(1):104-12; Xu et al. (1996) J Cell Biol, 132(1-2):239-49; Vlodaysky et al. (1990) Cancer Metastasis Rev, 9(3):203-2).

This Example studies the combination treatment of TTFields and lenvatinib in an INOVITRO™ 3D model (Novocure GmbH, Root, Switzerland) with collagen and cancer cells grown as a 3D structure. This Example also studies the combination treatment of TTFields and lenvatinib in an INOVIVO™ system (Novocure GmbH, Root, Switzerland; see, for example, Davidi (2020) Neuro-Oncology, 22(Supplement 2):ii104).

Experiments are performed to assess cell and tumor growth (cell count or 3D image capturing INOVITRO™, tumor growth INOVIVO™ monitoring), apoptosis (INOVITRO™-AnnexinV/PI staining and INOVIVO™-Ki67 tissue staining), secretion of cytokines and chemokines (in lysates derived from tumors and also conditioned media and serums examined by ELISA, cytokine array, multiplex assay, or luminex assay), collagen expression (by collagen staining and secretion), and vascular formation-angiogenesis of the tissue (by CD31 staining).

Cell lines used are either for cancers that are typically treated with lenvatinib (such as, but not limited to, differentiated thyroid cancer (DTC), advanced renal cell carcinoma (RCC), and/or hepatocellular carcinoma) and/or cancer cell lines that are typically treated with TTFields (such as, but not limited to, glioblastoma or pleural mesothelioma).

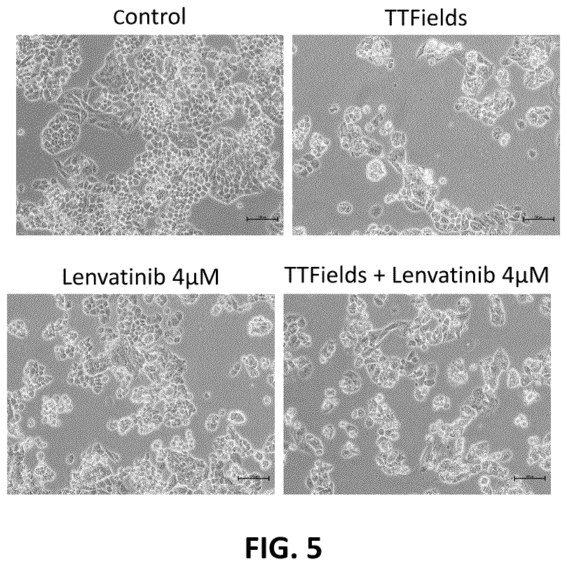

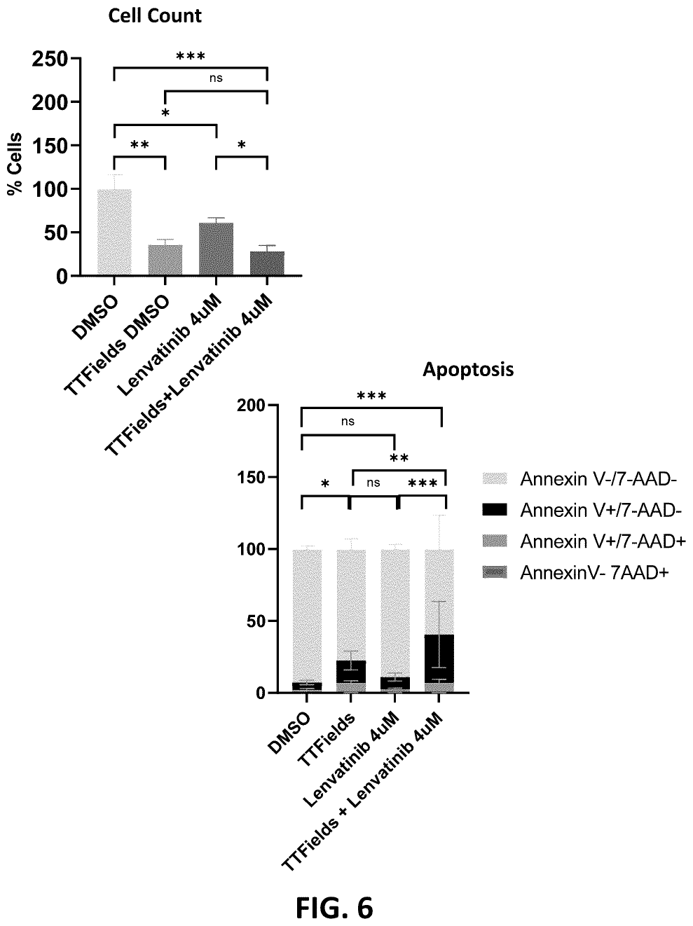

In particular, the combinatorial effect of TTFields and lenvatinib was examined in an ovarian carcinoma cell line (A2780). The effects of the concomitant therapy on cell count and apoptosis are shown in FIGS. 5-6. As can be seen, the combination of TTFields and lenvatinib provided an enhanced effect over either therapy alone.

Thus, the use of concomitant therapy that includes TTFields and lenvatinib has a synergistic effect. The combined TTFields/lenvatinib therapy is more effective at reducing viability of cancer cells and reducing tumor volume and/or preventing an increase in tumor volume when compared to either treatment alone. In addition, the use of TTFields in combination with lenvatinib reduces the amount of lenvatinib required to produce therapeutic results; when lenvatinib is combined with TTFields, the therapeutically effective concentration of lenvatinib is reduced by at least about 50% when compared to a dosage of lenvatinib known to be therapeutically effective in the absence of an alternating electric field.

### Non-Limiting Illustrative Embodiments of the Inventive Concept(s)

Illustrative embodiment 1. A method of reducing viability of cancer cells, the method comprising the steps of: (1) administering at least one composition to the cancer cells, wherein the at least one composition comprises at least one small molecule anti-angiogenic agent that specifically [physically] interacts with either vascular endothelial growth factor (VEGF) or a VEGF receptor (VEGFR) and selectively inhibits interaction between VEGF and VEGFR and/or inhibits VEGFR signal transduction at an IC₅₀ of less than about 10 nmol/L; and (2) applying an alternating electric field to the cancer cells for a period of time.
Illustrative embodiment 2. The method of illustrative embodiment 1, wherein the method is performed in vitro or ex vivo.
Illustrative embodiment 3. The method of illustrative embodiment 1, wherein the method is performed in vivo.
Illustrative embodiment 4. The method of any one of illustrative embodiments 1-3, wherein the cancer cells are selected from the group consisting of hepatocellular carcinoma cells, glioblastoma cells, pleural mesothelioma cells, differentiated thyroid cancer cells, advanced renal cell carcinoma cells, ovarian cancers, pancreatic cancers, lung cancer cells, breast cancer cells, and combinations thereof.
Illustrative embodiment 5. A method of treating cancer in a subject, the method comprising the steps of: (1) administering at least one composition to the subject, wherein the at least one composition comprises at least one small molecule anti-angiogenic agent that specifically interacts with either vascular endothelial growth factor (VEGF) or a VEGF receptor (VEGFR) and selectively inhibits interaction between VEGF and VEGFR and/or inhibits VEGFR signal transduction at an IC 50 of less than about 10 nmol/L; and (2) applying an alternating electric field to a target region of the subject.
Illustrative embodiment 6. A method of reducing a volume of a tumor and/or preventing an increase of volume of the tumor, wherein the tumor is present in a body of a living subject and includes a plurality of cancer cells, the method comprising the steps of: (1) administering at least one composition to the subject, wherein the at least one composition comprises at least one small molecule anti-angiogenic agent that specifically interacts with either vascular endothelial growth factor (VEGF) or a VEGF receptor (VEGFR) and selectively inhibits interaction between VEGF and VEGFR and/or inhibits VEGFR signal transduction at an IC₅₀ of less than about 10 nmol/L; and (2) applying an alternating electric field to a target region of the subject.
Illustrative embodiment 7. The method of illustrative embodiment 5 or 6, wherein the at least one composition is orally administered to the subject.
Illustrative embodiment 8. The method of any one of illustrative embodiments 5-7, wherein the cancer is selected from the group consisting of hepatocellular carcinoma, glioblastoma, pleural mesothelioma, differentiated thyroid cancer, advanced renal cell carcinoma, ovarian cancer, pancreatic cancer, lung cancer, breast cancer, and combinations thereof.
Illustrative embodiment 9. The method of any one of illustrative embodiments 1-8, wherein the at least one small molecule anti-angiogenic agent specifically interacts with at least one of VEGF-A, VEGF-B, VEGF-C, or VEGF-D.
Illustrative embodiment 10. The method of any one of illustrative embodiments 1-9, wherein the at least one small molecule anti-angiogenic agent that specifically interacts with at least one of VEGFR-1, VEGFR-2, or VEGFR-3.
Illustrative embodiment 11. The method of any of illustrative embodiments 1-10, wherein the at least one small molecule anti-angiogenic agent is selected from the group consisting of lenvatinib, axitinib, regorafenib, cabozantinib, anlotinib, pazopanib, albendazole, lucitanib, motesanib, aflibercept, ponatinib, cediranib, tivozanib, telatinib, apatinib, semaxanib, fruquintinib, lucitanib, anlotinib, taxifolin, sulfatinib, dovitinib, ningetinib, AZD2932, LY2874455, MGCD-265 analog, ZM 306416, ZM 323881, KRN 633, YF-452, ODM-203, AEE 788, BMS 605541, MAZ51, Ki 8751, SU 5402, SU 5408, SU5205, SU5214, SU 6668, SU 14813, XL 092, XL 184, BAW2881, BFH772, A-13958, SKLB1002, WAY-340935, ZD-4190, hVEGF-IN-1, R1530, VEGF-Grab, Soluble Vascular Endothelial Growth Factor Decoy Receptor FP3, VEGF decoy receptor fusion protein, a decoy receptor for VEGF, and combinations thereof.
Illustrative embodiment 12. The method of any one of illustrative embodiments 1-11, wherein the at least one small molecule anti-angiogenic agent is a Type I VEGFR inhibitor.
Illustrative embodiment 13. The method of illustrative embodiment 12, wherein the Type I VEGFR inhibitor is selected from the group consisting of pazopanib, axitinib, ponatinib, motesanib, and combinations thereof.
Illustrative embodiment 14. The method of any one of illustrative embodiments 1-13, wherein the at least one small molecule anti-angiogenic agent is a Type II VEGFR inhibitor.
Illustrative embodiment 15. The method of illustrative embodiment 14, wherein the Type II VEGFR inhibitor is selected from the group consisting of carbozantinib, lenvatinib, regorafenib, lucitanib, and combinations thereof.
Illustrative embodiment 16. The method of any one of illustrative embodiments 1-15, wherein the at least one small molecule anti-angiogenic agent is a Type III VEGFR inhibitor.
Illustrative embodiment 17. A method of reducing viability of cancer cells, and/or treating cancer in a subject, and/or reducing a volume of a tumor and/or preventing an increase of volume of the tumor, the method comprising the steps of: (1) administering at least one composition to the cancer cells/subject, wherein the at least one composition comprises at least one small molecule anti-angiogenic agent that is a selective inhibitor of vascular endothelial growth factor (VEGF) or a VEGF receptor (VEGFR) that specifically and solely binds to VEGF or VEGFR and does not bind to other targets; and (2) applying an alternating electric field to the cancer cells/target region of the subject for a period of time.
Illustrative embodiment 18. The method of illustrative embodiment 17, wherein the method is performed in vitro or ex vivo.
Illustrative embodiment 19. The method of illustrative embodiment 17, wherein the method is performed in vivo.
Illustrative embodiment 20. The method of any one of illustrative embodiments 17-19, wherein the cancer/cancer cells are selected from the group consisting of hepatocellular carcinoma cells, glioblastoma cells, pleural mesothelioma cells, differentiated thyroid cancer cells, advanced renal cell carcinoma cells, ovarian cancers, pancreatic cancers, lung cancer cells, breast cancer cells, and combinations thereof.
Illustrative embodiment 21. The method of any of illustrative embodiments 17-20, wherein the at least one small molecule anti-angiogenic agent is aflibercept.
Illustrative embodiment 22. A method of reducing viability of cancer cells, the method comprising the steps of: (1) administering at least one composition to the cancer cells, wherein the at least one composition comprises lenvatinib; and (2) applying an alternating electric field to the cancer cells for a period of time.
Illustrative embodiment 23. The method of illustrative embodiment 22, wherein the method is performed in vitro or ex vivo.
Illustrative embodiment 24. The method of illustrative embodiment 22, wherein the method is performed in vivo.
Illustrative embodiment 25. The method of any one of illustrative embodiments 22-24, wherein the cancer/cancer cells are selected from the group consisting of hepatocellular carcinoma cells, glioblastoma cells, pleural mesothelioma cells, differentiated thyroid cancer cells, advanced renal cell carcinoma cells, ovarian cancers, pancreatic cancers, lung cancer cells, breast cancer cells, and combinations thereof.
Illustrative embodiment 26. A method of treating cancer in a subject, the method comprising the steps of: (1) administering at least one composition to the subject, wherein the at least one composition comprises lenvatinib; and (2) applying an alternating electric field to a target region of the subject.
Illustrative embodiment 27. A method of reducing a volume of a tumor and/or a method of preventing an increase of volume of a tumor, wherein the tumor is present in a body of a living subject and includes a plurality of cancer cells, the method comprising the steps of: (1) administering at least one composition to the subject, wherein the at least one composition comprises lenvatinib; and (2) applying an alternating electric field to a target region of the subject.
Illustrative embodiment 28. The method of illustrative embodiment 26 or 27, wherein the at least one composition is orally administered to the subject.
Illustrative embodiment 29. The method of any of illustrative embodiments 26-28, wherein the composition comprising lenvatinib is administered to the cancer cells at a therapeutically effective concentration of lenvatinib.
Illustrative embodiment 30. The method of illustrative embodiment 29, wherein the therapeutically effective concentration of lenvatinib is reduced by at least about 50% with respect to a dosage of lenvatinib known to be therapeutically effective in the absence of an alternating electric field.
Illustrative embodiment 31. The method of illustrative embodiment 29 or 30, wherein the therapeutically effective concentration of lenvatinib is from about 12.5 nM to about 100 nM.
Illustrative embodiment 32. The method of any of illustrative embodiments 1-31, wherein at least one of: the alternating electric field is applied at a frequency in a range of from about 50 kHz to about 1 MHz; the alternating electric field has a field strength of at least about 1 V/cm in at least a portion of the cancer cells; and the period of time that the alternating electric field is applied is at least about 24 hours.
Illustrative embodiment 33. The method of any one of illustrative embodiments 1-32, wherein steps (1) and (2) are performed substantially simultaneously.
Illustrative embodiment 34. The method of any one of illustrative embodiments 1-32, wherein steps (1) and (2) are performed wholly or partially sequentially, and wherein the at least one composition is administered before the application of the alternating electric field has begun.
Illustrative embodiment 35. The method of any one of illustrative embodiments 1-33, wherein steps (1) and (2) are performed wholly or partially sequentially, and wherein the at least one composition is administered after the application of the alternating electric field has begun.
Illustrative embodiment 36. The method of illustrative embodiment 35, wherein the at least one composition is administered at least 24 hours after application of the alternating electric field has begun.
Illustrative embodiment 37. The method of illustrative embodiment 36, wherein the at least one composition is administered before the period of time the alternating electric field is applied has elapsed.
Illustrative embodiment 38. The method of illustrative embodiment 36, wherein the at least one composition is administered after the period of time has elapsed.
Illustrative embodiment 39. The method of illustrative embodiment 38, wherein the at least one composition is administered within about 96 hours of when the period of time elapsed.
Illustrative embodiment 40. The method of any of illustrative embodiments 1-39, wherein the period of time that the alternating electric field is applied is in a range of from about 24 hours to about 72 hours.
Illustrative embodiment 41. The method of any of illustrative embodiments 1-40, wherein the method further comprises the step of discontinuing the application of the alternating electric field.
Illustrative embodiment 42. The method of any of illustrative embodiments 1-41, wherein the at least one composition further comprises a pharmaceutically acceptable carrier.
Illustrative embodiment 43. The method of any one of illustrative embodiments 1-42, wherein steps (1) and (2) are repeated one or more times.
Illustrative embodiment 44. The method of any one of illustrative embodiments 1-43, wherein the method comprises the step of applying a first alternating electric field to the cancer cells/target region of the subject for a period of time and the step of applying a second alternating electric field to the cancer cells/target region of the subject for a period of time, and wherein the first and second alternating electric fields have different frequencies.
Illustrative embodiment 45. The method of illustrative embodiment 44, wherein the first frequency affects cell permeability, and wherein the second frequency affects cell viability.
Illustrative embodiment 46. The method of any of illustrative embodiments 1-45, wherein the at least one composition further comprises at least one additional therapeutic agent.
Illustrative embodiment 47. The method of any of illustrative embodiments 1-46, wherein the method further comprises the step of administering a second composition to the cancer cells, wherein the second composition comprises at least one additional therapeutic agent, and wherein the first and second compositions are administered substantially simultaneously or wholly or partially sequentially.
Illustrative embodiment 48. The method of illustrative embodiment 46 or 47, wherein the at least one additional therapeutic agent is selected from the group consisting of a checkpoint immune inhibitor, an anti-PD-1 therapeutic agent, a chemotherapeutic agent, a TKI inhibitor, an mTOR inhibitor, an Akt inhibitor, an aromatase inhibitor, a radiotherapy agent, a biologic, and combinations thereof.
Illustrative embodiment 49. The method of illustrative embodiment 48, wherein the at least one additional therapeutic agent is selected from the group consisting of Pembrolizumab, Tislelizumab, Paclitaxel, Docetaxel, Ifosamide, Etoposide (Vepesid), Gemcitabine, Cardoplatin, Everolimus, Letrozole, Denosumab, and combinations thereof.
Illustrative embodiment 50. The method of any of illustrative embodiments 1-49, further comprising the step of administering at least one additional therapy to the cells/subject.
Illustrative embodiment 51. The method of illustrative embodiment 50, wherein the at least one additional therapy is selected from the group consisting of radiation therapy, photodynamic therapy, transarterial chemoembolization (TACE), and combinations thereof.
Illustrative embodiment 52. A composition for use in the methods of any of illustrative embodiments 1-16 and 32-51 (when dependent upon illustrative embodiments 1-16), wherein the composition comprises at least one small molecule anti-angiogenic agent that specifically interacts with either vascular endothelial growth factor (VEGF) or a VEGF receptor (VEGFR) and selectively inhibits interaction between VEGF and VEGFR and/or inhibits VEGFR signal transduction at an IC₅₀ of less than about 10 nmol/L.
Illustrative embodiment 53. Use of a composition in the methods of any of illustrative embodiments 1-16 and 32-51 (when dependent upon illustrative embodiments 1-16), wherein the composition comprises at least one small molecule anti-angiogenic agent that specifically interacts with either vascular endothelial growth factor (VEGF) or a VEGF receptor (VEGFR) and selectively inhibits interaction between VEGF and VEGFR and/or inhibits VEGFR signal transduction at an IC₅₀ of less than about 10 nmol/L.
Illustrative embodiment 54. A composition for use in the methods of any of illustrative embodiments 17-21 and 32-51 (when dependent upon illustrative embodiments 17-21), wherein the composition comprises at least one small molecule anti-angiogenic agent that is a selective inhibitor of vascular endothelial growth factor (VEGF) or a VEGF receptor (VEGFR) that specifically and solely binds to VEGF or VEGFR and does not bind to other targets.
Illustrative embodiment 55. Use of a composition in the methods of any of illustrative embodiments 17-21 and 32-51 (when dependent upon illustrative embodiments 17-21), wherein the composition comprises at least one small molecule anti-angiogenic agent that is a selective inhibitor of vascular endothelial growth factor (VEGF) or a VEGF receptor (VEGFR) that specifically and solely binds to VEGF or VEGFR and does not bind to other targets.
Illustrative embodiment 56. A composition for use in the methods of any of illustrative embodiments 22-31 and 32-51 (when dependent upon illustrative embodiments 22-31), wherein the composition comprises lenvatinib.
Illustrative embodiment 57. Use of a composition in the methods of any of illustrative embodiments 22-31 and 32-51 (when dependent upon illustrative embodiments 22-31), wherein the composition comprises lenvatinib.
Illustrative embodiment 58. A kit comprising: at least one pair of transducer arrays for generating an alternating electric field there between upon application of the transducer arrays to at least one cell and/or placement of the transducer arrays on a subject; and at least one composition of any of illustrative embodiments 52, 54, or 56.

While the attached disclosures describe the inventive concept(s) in conjunction with the specific experimentation, results, and language set forth hereinafter, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A method of reducing viability of cancer cells, the method comprising the steps of:
(1) administering at least one composition to the cancer cells, wherein the at least one composition comprises at least one small molecule anti-angiogenic agent that specifically interacts with either a vascular endothelial growth factor (VEGF) or a VEGF receptor (VEGFR) and selectively inhibits interaction between VEGF and VEGFR and/or inhibits VEGFR signal transduction at an IC₅₀ of less than about 10 nmol/L; and
(2) applying an alternating electric field to the cancer cells for a period of time.

2. The method of claim 1, wherein at least one of:
the alternating electric field is applied at a frequency in a range of from about 50 kHz to about 1 MHz;
the alternating electric field has a field strength of at least about 1 V/cm in at least a portion of the cancer cells; and
the period of time that the alternating electric field is applied is at least about 24 hours.

3. The method of claim 1, wherein the at least one small molecule anti-angiogenic agent is selected from the group consisting of lenvatinib, axitinib, regorafenib, cabozantinib, anlotinib, pazopanib, albendazole, lucitanib, motesanib, aflibercept, ponatinib, cediranib, tivozanib, telatinib, apatinib, semaxanib, fruquintinib, lucitanib, anlotinib, taxifolin, sulfatinib, dovitinib, ningetinib, AZD2932, LY2874455, MGCD-265 analog, ZM 306416, ZM 323881, KRN 633, YF-452, ODM-203, AEE 788, BMS 605541, MAZ51, Ki 8751, SU 5402, SU 5408, SU5205, SU5214, SU 6668, SU 14813, XL 092, XL 184, BAW2881, BFH772, A-13958, SKLB1002, WAY-340935, ZD-4190, hVEGF-IN-1, R1530, VEGF-Grab, Soluble Vascular Endothelial Growth Factor Decoy Receptor FP3, VEGF decoy receptor fusion protein, a decoy receptor for VEGF, and combinations thereof.

4. The method of claim 1, wherein steps (1) and (2) are performed substantially simultaneously.

5. The method of claim 1, wherein steps (1) and (2) are performed wholly or partially sequentially, and wherein the at least one composition is administered before the application of the alternating electric field has begun.

6. The method of claim 1, wherein steps (1) and (2) are performed wholly or partially sequentially, and wherein the at least one composition is administered after the application of the alternating electric field has begun.

7. The method of claim 1, wherein steps (1) and (2) are repeated one or more times.

8. The method of claim 1, wherein the cancer cells are selected from the group consisting of hepatocellular carcinoma cells, glioblastoma cells, pleural mesothelioma cells, differentiated thyroid cancer cells, advanced renal cell carcinoma cells, ovarian cancers, pancreatic cancers, lung cancer cells, breast cancer cells, and combinations thereof.

9. A method of treating cancer in a subject, the method comprising the steps of:
(1) administering at least one composition to the subject, wherein the at least one composition comprises at least one small molecule anti-angiogenic agent that specifically interacts with either a vascular endothelial growth factor (VEGF) or a VEGF receptor (VEGFR) and selectively inhibits interaction between VEGF and VEGFR and/or inhibits VEGFR signal transduction at an IC₅₀ of less than about 10 nmol/L; and
(2) applying an alternating electric field to a target region of the subject.

10. The method of claim 9, wherein the at least one small molecule anti-angiogenic agent is selected from the group consisting of lenvatinib, axitinib, regorafenib, cabozantinib, anlotinib, pazopanib, albendazole, lucitanib, motesanib, aflibercept, ponatinib, cediranib, tivozanib, telatinib, apatinib, semaxanib, fruquintinib, lucitanib, anlotinib, taxifolin, sulfatinib, dovitinib, ningetinib, AZD2932, LY2874455, MGCD-265 analog, ZM 306416, ZM 323881, KRN 633, YF-452, ODM-203, AEE 788, BMS 605541, MAZ51, Ki 8751, SU 5402, SU 5408, SU5205, SU5214, SU 6668, SU 14813, XL 092, XL 184, BAW2881, BFH772, A-13958, SKLB1002, WAY-340935, ZD-4190, hVEGF-IN-1, R1530, VEGF-Grab, Soluble Vascular Endothelial Growth Factor Decoy Receptor FP3, VEGF decoy receptor fusion protein, a decoy receptor for VEGF, and combinations thereof.

11. The method of claim 9, wherein the at least one composition is orally administered to the subject.

12. The method of claim 9, wherein at least one of:
the alternating electric field is applied at a frequency in a range of from about 50 kHz to about 1 MHz;
the alternating electric field has a field strength of at least about 1 V/cm in at least a portion of the cancer cells; and
the period of time that the alternating electric field is applied is at least about 24 hours.

13. The method of claim 9, wherein steps (1) and (2) are performed substantially simultaneously.

14. The method of claim 9, wherein steps (1) and (2) are performed wholly or partially sequentially, and wherein the at least one composition is administered before the application of the alternating electric field has begun.

15. The method of claim 9, wherein steps (1) and (2) are performed wholly or partially sequentially, and wherein the at least one composition is administered after the application of the alternating electric field has begun.

16. The method of claim 9, wherein steps (1) and (2) are repeated one or more times.

17. The method of claim 9, wherein the cancer is selected from the group consisting of hepatocellular carcinoma, glioblastoma, pleural mesothelioma, differentiated thyroid cancer, advanced renal cell carcinoma, ovarian cancer, pancreatic cancer, lung cancer, breast cancer, and combinations thereof.

18. A method of reducing a volume of a tumor and/or preventing an increase of volume of the tumor, wherein the tumor is present in a body of a living subject and includes a plurality of cancer cells, the method comprising the steps of:
(1) administering at least one composition to the subject, wherein the at least one composition comprises at least one small molecule anti-angiogenic agent that specifically interacts with either a vascular endothelial growth factor (VEGF) or a VEGF receptor (VEGFR) and selectively inhibits interaction between VEGF and VEGFR and/or inhibits VEGFR signal transduction at an IC₅₀ of less than about 10 nmol/L; and
(2) applying an alternating electric field to a target region of the subject.

19. The method of claim 18, wherein the at least one small molecule anti-angiogenic agent is selected from the group consisting of lenvatinib, axitinib, regorafenib, cabozantinib, anlotinib, pazopanib, albendazole, lucitanib, motesanib, aflibercept, ponatinib, cediranib, tivozanib, telatinib, apatinib, semaxanib, fruquintinib, lucitanib, anlotinib, taxifolin, sulfatinib, dovitinib, ningetinib, AZD2932, LY2874455, MGCD-265 analog, ZM 306416, ZM 323881, KRN 633, YF-452, ODM-203, AEE 788, BMS 605541, MAZ51, Ki 8751, SU 5402, SU 5408, SU5205, SU5214, SU 6668, SU 14813, XL 092, XL 184, BAW2881, BFH772, A-13958, SKLB1002, WAY-340935, ZD-4190, hVEGF-IN-1, R1530, VEGF-Grab, Soluble Vascular Endothelial Growth Factor Decoy Receptor FP3, VEGF decoy receptor fusion protein, a decoy receptor for VEGF, and combinations thereof.

20. The method of claim 18, wherein at least one of:
the alternating electric field is applied at a frequency in a range of from about 50 kHz to about 1 MHz;
the alternating electric field has a field strength of at least about 1 V/cm in at least a portion of the cancer cells; and
the period of time that the alternating electric field is applied is at least about 24 hours.
